(19) United States
(12) Patent Application Publication
(10) Pub. No.: US 2024/0139417 A1
(43) Pub. Date: May 2, 2024

(54) SYRINGE CARRIER

(71) Applicant: SHL MEDICAL AG, ZUG (CH)

(72) Inventors: Chia-Hsin Su, New Taipei City (TW); Nurettin Ali, Boca Raton, FL (US); Antonio Farieta, Baar (CH); Meng-Jhen Chiou, Taoyuan City (TW); Pei Yu Chao, Taoyuan City (TW); Jason Mondro, Deerfield Beach, FL (US)

(21) Appl. No.: 18/275,340

(22) PCT Filed: Feb. 4, 2022
(86) PCT No.: PCT/EP2022/052684

§ 371 Date: Aug. 1, 2023

Related U.S. Application Data
(60) Provisional application No. 63/149,380, filed on Feb. 15, 2021.
(60) Provisional application No. 63/153,408, filed on Feb. 25, 2021.
(60) Provisional application No. 63/158,580, filed on Mar. 9, 2021.
(60) Provisional application No. 63/162,601, filed on Mar. 18, 2021.
(60) Provisional application No. 63/178,577, filed on Apr. 23, 2021.
(60) Provisional application No. 63/287,644, filed on Dec. 9, 2021.

(30) Foreign Application Priority Data
Oct. 6, 2021 (EP) 21201195.1

(51) Int. Cl. A61M 5/24; A61M 5/32

(52) U.S. Cl. CPC A61M 5/24; A61M 5/3202

(57) ABSTRACT

The application describes syringe carriers for medicament delivery devices such as autoinjectors. In particular, a medicament delivery device sub-assembly is described. The medicament delivery device sub-assembly has a housing extending along a longitudinal axis from a proximal end to a distal end, the housing has a tubular section with an internal surface facing towards the axis and an external surface facing away from the axis, wherein the housing has an aperture extending through the tubular section from the internal surface to the external surface; and a housing cover assembly attached in the aperture of the housing, the housing cover assembly being configured to secure a syringe in place relative to the housing.

### CROSS REFERENCE TO RELATED APPLICATIONS

The disclosure of each of the following applications is incorporated herein by reference: U.S. provisional patent application 63/149,380, filed 15 Feb. 2021 by SHL Medical AG; U.S. provisional patent application 63/153,408, filed 25 Feb. 2021 by SHL Medical AG; U.S. provisional patent application 63/158,580, filed 9 Mar. 2021 by SHL Medical AG; U.S. provisional patent application 63/162,601, filed 18 Mar. 2021 by SHL Medical AG; U.S. provisional patent application 63/178,577, filed 23 Apr. 2021 by SHL Medical AG.

### TECHNICAL FIELD

Syringe carriers for medicament delivery devices such as autoinjectors.

### BACKGROUND

Medicament delivery devices such as autoinjectors often have a syringe carrier to support a syringe (typically a glass syringe) within the medicament delivery device. Although existing syringe carriers can be effective at supporting the syringe, the inventors have appreciated that further improvements can be made.

### SUMMARY OF THE INVENTION

The invention concerns a number of different concepts for supporting a syringe in a medicament delivery device as described below.

The invention is defined by the appended claims, to which reference should now be made.

An aspect concerns a medicament delivery device sub-assembly, the medicament delivery device sub-assembly comprising: a housing extending along a longitudinal axis from a proximal end to a distal end, the housing comprising a tubular section with an internal surface facing towards the axis and an external surface facing away from the axis, wherein the housing comprises an aperture extending through the tubular section from the internal surface to the external surface; and a housing cover assembly attached in the aperture of the housing, the housing cover assembly being configured to hold a syringe in place relative to the housing. This can allow a syringe (or another type of medicament-containing component such as a cartridge) to be inserted and held (secured) inside a medicament delivery device without requiring any components to bend during medicament delivery device assembly. In particular, this can be useful for holding a syringe with a rigid needle shield, especially when the rigid needle shield is as wide (or wider than) the medicament holder of the syringe.

Optionally, the housing cover assembly comprises a protrusion extending towards the longitudinal axis. Optionally, the protrusion of the housing cover assembly comprises a surface facing the distal end of the housing, wherein the surface is shaped to fit between a medicament delivery shield and a shoulder of said syringe so as to support said shoulder of said syringe.

Optionally, the medicament delivery device sub-assembly comprises a syringe in the housing, and wherein the syringe is held relative to the housing by the housing cover assembly.

Optionally, the housing cover assembly consists of two housing covers arranged opposite each other relative to the axis. Optionally, the housing cover assembly comprises two separate housing covers. Optionally, the housing cover assembly is a single integral part.

Optionally, the housing cover assembly comprises an arm. Optionally, the arm extends in the direction of the axis. Optionally, the arm is rigid. Optionally, the protrusion of the housing cover assembly is attached to the arm.

Optionally, the external surface of the tubular section is configured to be held by a user during use of a medicament delivery device comprising the medicament delivery device sub-assembly.

Optionally, the housing cover assembly comprises a window. This can allow users to view a medicament in a medicament delivery device. Optionally, the housing cover assembly comprises a window frame extending around the window. This can provide an anti-roll feature.

Optionally, the housing cover assembly comprises at least one pin extending perpendicular to the longitudinal axis. Optionally, the pin is arranged so that the pin is between a syringe and a syringe carrier in a completed medicament delivery device. Optionally, the pin is arranged so that the pin is between a shoulder of the syringe and the syringe carrier in a completed medicament delivery device.

Optionally, the housing cover assembly comprises an insert and the housing comprises a flexible arm adjacent to the insert. Optionally, the housing cover assembly is configured to indirectly hold a syringe in place relative to the housing. Optionally, the housing cover assembly is configured to directly hold a syringe in place relative to the housing. Optionally, the housing cover assembly is configured to hold a syringe in place relative to the housing so that the syringe cannot move in the proximal direction relative to the housing.

An aspect concerns a medicament delivery device comprising the sub-assembly of any previous claim. Optionally, the medicament delivery device is an autoinjector. Optionally, the medicament delivery device comprises a syringe, and a shoulder of the syringe is supported directly or indirectly by the housing cover assembly. Optionally, the medicament delivery device comprises a syringe carrier.

An aspect concerns a method of assembling a medicament delivery device sub-assembly or of assembling a medicament delivery device, the method comprising the steps of: providing a syringe, a housing comprising an aperture, and a housing cover assembly comprising a housing cover, the housing cover comprising a protrusion configured to support the syringe; inserting the syringe into the housing; and attaching a housing cover assembly in the aperture of the housing so that the protrusion of the housing cover assembly supports the syringe.

### BRIEF DESCRIPTION OF THE DRAWINGS

Embodiments of the present disclosure will now be described by way of example only and with reference to the accompanying drawings as listed below.

FIG. 1 shows a cross-section view of a housing and a housing cover assembly.

FIG. 2 shows a cross-section view of a syringe.

FIG. 3 shows a cross-section view of a housing and a syringe.

FIG. 4 shows a cross-section view of a housing, a housing cover assembly and a syringe.

FIG. 5 shows a perspective view of part of a medicament delivery device, including a housing cover assembly.

FIG. 6 shows a side view of the housing cover assembly of FIG. 5.

FIG. 7 shows another side view of part of the housing cover assembly of FIG. 5.

FIGS. 8 to 10 show perspective views of a syringe carrier.

FIGS. 11 and 12 show perspective views of a proximal part of the syringe carrier of FIG. 8.

FIGS. 13 and 14 show the syringe carrier of FIG. 8 during assembly into a medicament delivery device.

FIG. 15 shows an assembled medicament delivery device including the syringe carrier of FIG. 8.

FIGS. 16 to 22 show various views of another syringe carrier.

FIG. 23 shows the syringe carrier of FIG. 16 holding a syringe.

FIG. 24 shows another syringe carrier holding a syringe.

FIG. 25 shows the separate parts of the syringe carrier of FIG. 24.

FIG. 26 shows a side view of part of the syringe carrier of FIG. 24.

FIG. 27 shows views of parts of a syringe carrier.

FIG. 28 shows a syringe carrier that could include the parts shown in FIG. 27.

FIG. 29 shows a perspective view of an example autoinjector.

FIG. 3o shows a perspective view of an example syringe.

FIG. 31 shows a cross-section view of the example syringe of FIG. 30.

FIG. 32 shows a cross-section view of part of a medicament delivery device.

FIG. 33 shows a perspective view of part of the medicament delivery device shown in FIG. 32 during assembly.

FIG. 34 shows a cross-section view of part of a medicament delivery device.

FIG. 35 shows a perspective view of part of a housing.

FIG. 36 shows a side view of part of the housing of FIG. 35.

FIGS. 37 and 38 show perspective views of a housing cover.

FIG. 39 shows the housing of FIG. 34 with the housing cover of FIG. 37.

### DETAILED DESCRIPTION

Embodiments of the present disclosure will now be described by way of example only and with reference to the accompanying drawings. The invention is defined by the appended claims, to which reference should now be made.

In general, the present disclosure describes various syringe carriers for medicament delivery devices such as autoinjectors. Generally, these syringe carriers are for use in devices in which the syringe is inserted into the device (for example into a housing of the device) in the axial direction during assembly.

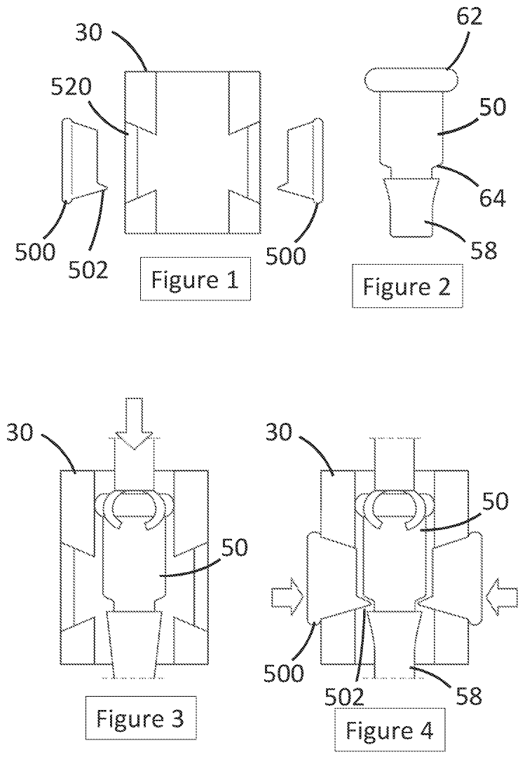

In one example, a syringe carrier that can subsequently be inserted into a housing is not used, and instead a housing cover (body cover) 500 is provided as a syringe carrier to support the syringe (FIGS. 1 to 4). More specifically, a housing (body) 30 with an aperture 520 (two apertures in this example) is provided, as shown in FIG. 1. A syringe is then inserted into the housing (FIG. 3). The housing cover is then attached (FIG. 4). The housing cover (or two housing covers in this example) is attached (for example by a snap fit, with ultrasonic welding or with glue) to the housing. The particular shape of the housing cover will depend on the shape of the syringe and of the housing, but in general the housing cover will comprise a protrusion or rib arranged to fit in the gap between the RNS 58 of the syringe and the syringe shoulder 64 to support the syringe 50. Movement of the syringe in the proximal direction relative to the housing is therefore limited by the housing cover. Optionally, movement of the syringe in the distal direction relative to the housing can be restricted by the housing or another component of the medicament delivery device.

In the example given in FIG. 1, a housing 30 comprises an aperture 520. In an initial assembly step, a syringe (such as the syringe in FIG. 2) is inserted into the housing as shown in FIG. 3. The syringe 50 is shown being inserted into the distal end of the housing 30, although the syringe could alternatively be inserted into the proximal end of the housing. Subsequently, the housing covers 500 are attached to the housing as shown in FIG. 4. When assembly is completed, the protrusions 502 of the housing covers 500 support the shoulder 64 of the syringe 50.

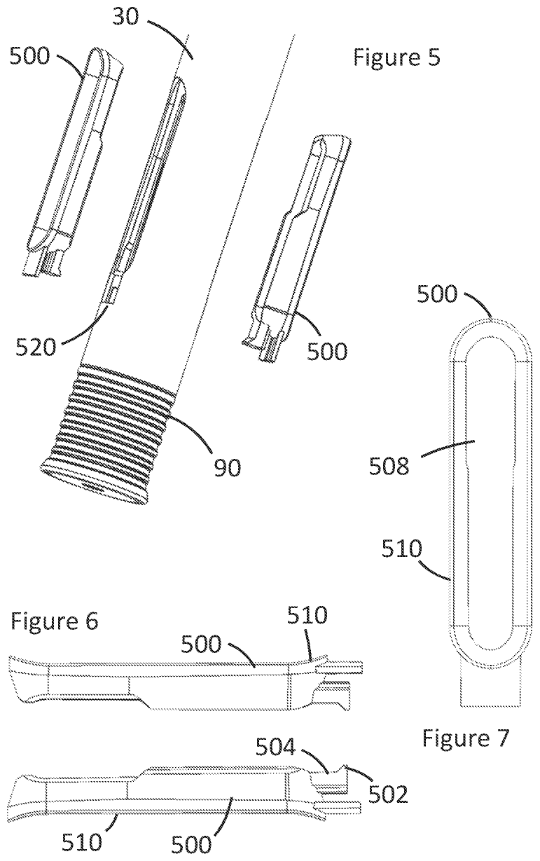

Another more detailed example is shown in FIGS. 5 to 7. A front portion of a sub-assembly can be seen, with an optional cap 90 already attached to a housing 30 (the cap could be attached before, during or after the assembly steps described herein). A housing cover assembly comprising two housing covers 500 is also visible, with the housing covers 500 shown spaced apart from the housing 30 (i.e. prior to assembly) in FIG. 5. A cut-out (aperture) 520 in the housing 30, which is where one of the housing covers 500 will be mounted (attached), can also be seen. The housing has a tubular section with an internal surface facing towards the axis and an external surface facing away from the axis. Typically, the external surface of the tubular section is configured to be held by a user during use of a medicament delivery device.

In FIGS. 5 to 7, three different views of the housing covers 500 can be seen. In this example, the two housing covers are identical, though this is optional. Each housing cover comprises an arm 504. The arm 504 extends in the axial direction, though it could alternatively extend in another direction, for example the circumferential direction. The arm 504 is optional, and the protrusion 502 could be attached directly to the rest of the housing cover. In this example, the arm is attached to the rest of the housing cover at the distal end of the arm, although alternatively another part of the arm such as the proximal end of the arm could be attached to the rest of the housing cover. The protrusion 502 is attached to the proximal end of the arm, although the protrusion could alternatively be attached elsewhere on the arm. Optionally, the arm is rigid, which can help the protrusion support the syringe effectively.

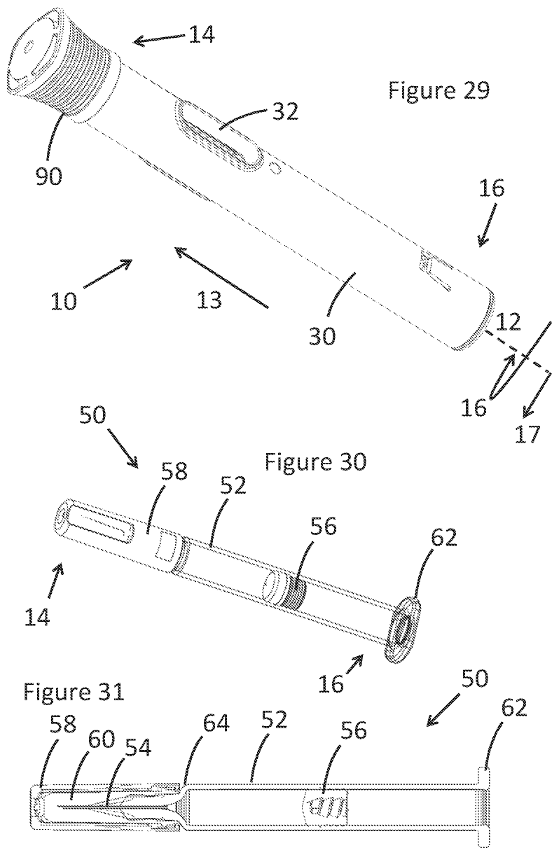

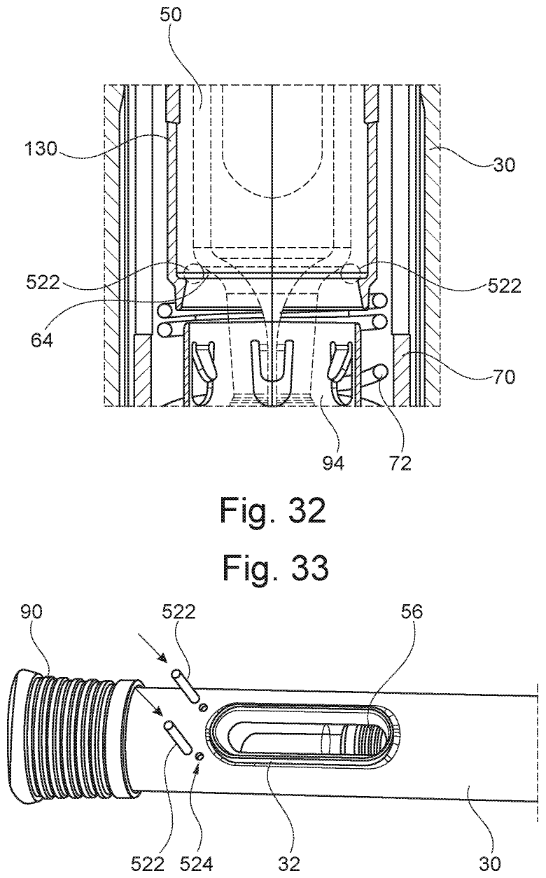

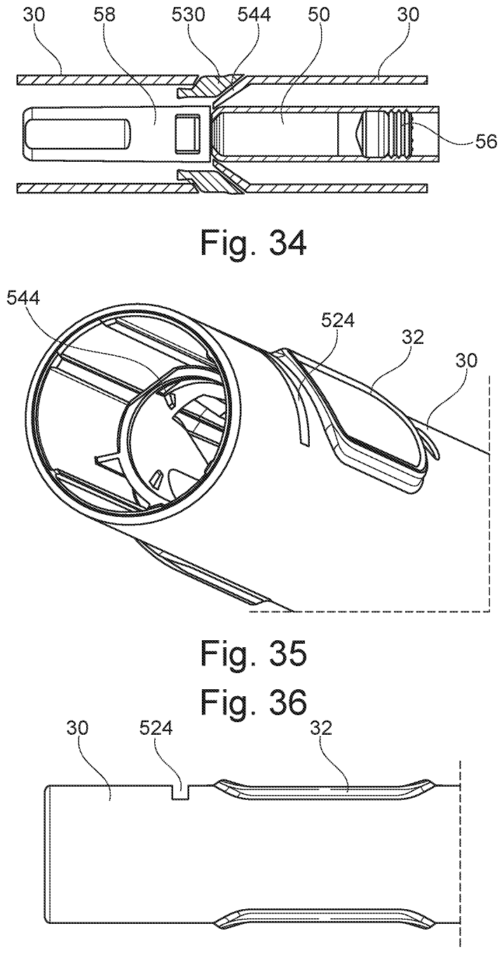

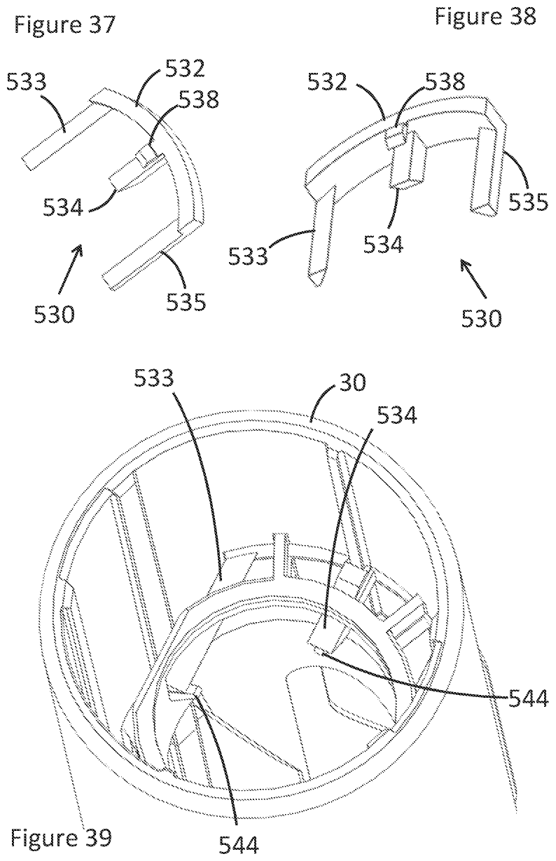

An optional window 508 is also provided in the housing cover, with an optional window frame 510 extending around the edge of the window cover. Provision of a window in the housing cover can allow a user to see the syringe before, during and/or after medicament delivery. When a window is needed, for example to meet regulatory requirements, it could be beneficial to provide the window in the housing cover rather than in the housing so as to reduce the number of holes in the housing and thereby increase the structural integrity of the housing. Provision of a window frame can help maximise the structural integrity of the housing cover. When the housing cover is assembled (attached to the housing), the window frame optionally extends further from the axis than the external surface of the housing—the window frame could have the same profile as shown in FIG. 29 with the window frame that is visible around the window 32, for example. The window frame can therefore also function as an anti-roll feature.

The two housing covers together make up a housing cover assembly. In the examples above, the housing cover assembly comprises two housing covers, and providing two housing covers that are opposite each other relative to the axis can be beneficial, for example in terms of balanced forces on the device. Alternatively, one, three or more covers could be provided in a housing cover assembly. Rather than two (or more) separate housing covers, the housing covers could be attached to each other, for example by a strip (optionally a flexible strip).

This would allow the two (or more) separate housing covers to be a single integral part, which could simplify assembly by reducing the number of separate parts. Although a housing cover needs to fit the aperture in the housing, the shape of both the housing cover and the aperture could be varied, depending for example on the shape of other medicament delivery device components. In examples with a window in the housing cover, the shape of the window can also be varied, for example based on the shape of the syringe and other medicament delivery device components, and on what needs to be visible within the medicament delivery device.

Two different shapes for the protrusion 502 are shown (see e.g. FIGS. 1 and 6), and these two protrusion shapes could both be used in both of the examples in FIGS. 1 and 6. The shape of the protrusion can be further varied, for example based on the shape of the shoulder of the syringe and/or based on the shape of the gap between the shoulder and the rigid needle shield of the syringe. In both FIG. 1 and FIG. 6, the distally facing surface of the protrusion is sloped (relative to a plane perpendicular to the longitudinal axis). This is optional, but can be beneficial, as this slope can be matched with the slope of the shoulder of the syringe, providing better support for the syringe by spreading the load of the syringe on the protrusion over a greater contact area.

Typically, the syringe is inserted into the housing (typically to its final position relative to the housing) before the housing covers are attached to the housing. However, the housing covers could initially be attached to the housing in a first position and then moved to a second position relative to the housing. For example, the first position could be a position where the protrusion 502 is further from the axis than in the second position. This could allow the housing covers to be attached to the housing (for example by a snap-fit or another fixture that can releasably hold the housing covers in the first position) before the syringe is inserted into the housing, and for the housing covers to be moved from the first position to the second position after the syringe is inserted into the housing, thereby moving the housing covers into a position where the housing covers support the syringe at the shoulder. In the second position, and more generally also in cases where the housing covers are directly attached in their final position relative to the housing, there are various suitable options for attaching the housing covers to the housing, for example with glue or snap-fit fixtures.

FIGS. 32 and 33 show an alternative approach to providing a housing cover. In this example, the housing cover comprises two inserts, in this case pins 522 (though one, three or more could alternatively be provided). The two pins are opposite each other relative to the axis 12 (see FIG. 29), and are therefore parallel to one another, though this is optional and the pins could be angled relative to one another. The pins are arranged between a syringe 50 and a syringe carrier 130 (the syringe carrier is a separate component in this example, but could alternatively be an integral part of a housing 30). The syringe carrier 130 can help support the pins (e.g. to stop the pins from bending under the force of a plunger rod pushing a stopper of the syringe during medicament delivery), although this is optional, and a syringe carrier 130 is therefore optional.

A distally facing surface of each pin is adjacent to (and typically touching) a shoulder 64 of the syringe 50. A proximally facing surface of each pin is adjacent to (and typically touching) a corresponding distally facing surface of the syringe carrier. As can be seen in FIG. 32, the distally facing surface of the pin would typically be angled to also face towards the axis 12, as the syringe shoulder is typically angled as well. Similarly, the proximally facing surface of the pin would typically be angled to also face away from the axis 12. A relevant factor here is that it is beneficial that the internal diameter of the syringe carrier is greater than the external diameter of a proximal part of the syringe (in particular greater than the external diameter of the RNS of the syringe), as this can simplify insertion of the syringe into the syringe carrier.

The provision of housing cover (pins in this example) allows this by supporting the syringe at the shoulder using the housing cover (pins in this example) rather than a feature of the syringe carrier itself.

As can be seen in FIG. 30, the pins 522 are inserted into the housing through holes in the housing. As a result, the pins would typically be immovable relative to the housing in a completed device. The pins could be held in place in the housing in various ways, for example by glue, by a press fit (in the hole in the housing or with another component), by a snap fit, and/or by a label on the outside of the housing. In a method of assembly of a device comprising the pins, the same approach can be taken as with the example given in FIGS. 5 to 7, so this will not be described separately. As with that method, the syringe could be moved slightly by the insertion of the pins (the housing cover) if the syringe is in the way of the pins when the pins are inserted.

For reference, a needle guard 70, a needle guard spring 72, and an RNS remover 94 are also shown in FIG. 32, although these components and their features are not essential to this particular example, as is also emphasised elsewhere in this application. Similarly, FIG. 33 also shows a cap 90, a window 32 of a housing 30, and part of a syringe through the window 32, including a stopper 56.

The cross-section of the pins is circular in the depicted example, but other shapes could be used, such as square, rectangular or an irregular shape. The provision of a syringe carrier is optional, as the pins can alternatively made sturdy enough to support the syringe without themselves being supported. In this example, the housing cover comprises only the pins, and no other features are provided. However, as in the example in FIGS. 5 to 7, the housing cover could comprise other features in addition to a pin or pins, such as features shown in FIGS. 5 to 7 (such as the window frame 510). As mentioned above, the depicted example comprises two pins but only a single pin could be used in an alternative example. One way of achieving this is to provide support for the shoulder of the syringe on one side (e.g. with a part of the syringe carrier, the housing, or another component, such as a distally facing surface of the syringe carrier or of the housing) so that the syringe is already supported on one side by another component. Providing one (or more) pins would then complete the support by providing support elsewhere around the circumference of the syringe shoulder. In an alternative example, the pins 522 could support the syringe 50 indirectly, for example by supporting flexible arms of the housing as shown in the examples in FIGS. 34 and 35. Rather than one or two pins, three or more pins could also be provided, for example spaced out equally around the circumference of the housing.

FIG. 34 shows another example, in which the housing cover, which in this case is an insert 530, supports a flexible arm 544 of the housing rather than directly supporting the syringe 50.

FIGS. 35 to 39 show another example similar to the example in FIG. 35. As can be seen in FIGS. 37 and 38 in particular, the insert 530 comprises a base 532 and three arms 533, 534, 535 extending from the base 532. A first end of each of the arms 533, 534, 535 is attached to the base 532. The second arm 534 is between the first arm 533 and the third arm 535. Each of the arms 533, 534, 535 has a triangular cross-section at the end distal from the base 532 (the second end). The triangular cross-section provides a face on the second end of the second arm 534 that is angled away from the base 532, whereas on the first arm 533 and the third arm 535, the triangular cross-section provides faces that are angled towards each other (and are angled neither towards nor away from the base 532). This results in a face on each arm that is angled towards the axis 12 when the insert is in place in a housing 30, as shown in FIG. 39. As a result, the faces on the three arms support the flexible arms 544 of the housing 30 when the insert 530 is in place in the housing 30.

The base 532 is typically, although not necessarily, curved to match the curvature of the housing 30. The insert could be held in place in the housing by, for example, glue, a press fit, by a snap fit, and/or by a label on the outside of the housing. In the depicted example in FIGS. 35 to 39, an optional arm 538 could help with holding the insert in place with a friction and/or snap fit.

FIGS. 34 to 39 show examples in which the insert indirectly supports the syringe (via a flexible arm of the housing). Alternatively, the insert 530 could support the syringe 50 directly, without a flexible arm of the housing being needed.

Various other structural variations are possible in the design described above. For example, the length of the first arm and third arm could be varied, along with the angle, shape and position of the flexible arms of the housing, with the angle and shape of the faces of on the second end of the first arm and the second end of the third arm being altered accordingly. The number of arms on the insert and the number of flexible arms on the housing can also be varied. In the design in FIGS. 35 to 39, a single insert is provided, though two or more inserts could alternatively be used, for example two or more inserts (for example two, three or four inserts) equally spaced out around the circumference of the housing, each with one or more arms, each arm supporting a one or more flexible arms of the housing. The housing cover would typically be rigid but could also be flexible or deformable, depending for example on the strength requirements for a particular medicament delivery device design and/or on shape of the other components the housing cover interacts with in a completed medicament delivery device.

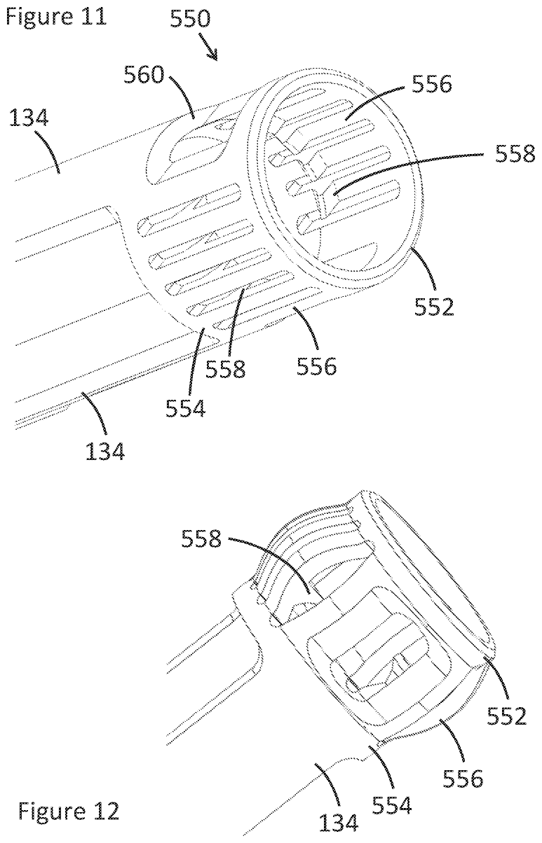

In another example, a syringe carrier (a proximal part of which is shown in FIG. 11) has a flexible portion that is in between the proximal end of the syringe carrier and the distal end of the syringe carrier (that is, the flexible portion is spaced apart from both the proximal end of the syringe carrier and from the distal end of the syringe carrier). The syringe carrier comprises flexible bridges (flexible strips) spaced around the circumference of the syringe carrier, and protrusions arranged on the bridges (strips). The protrusions can support the shoulder of a syringe.

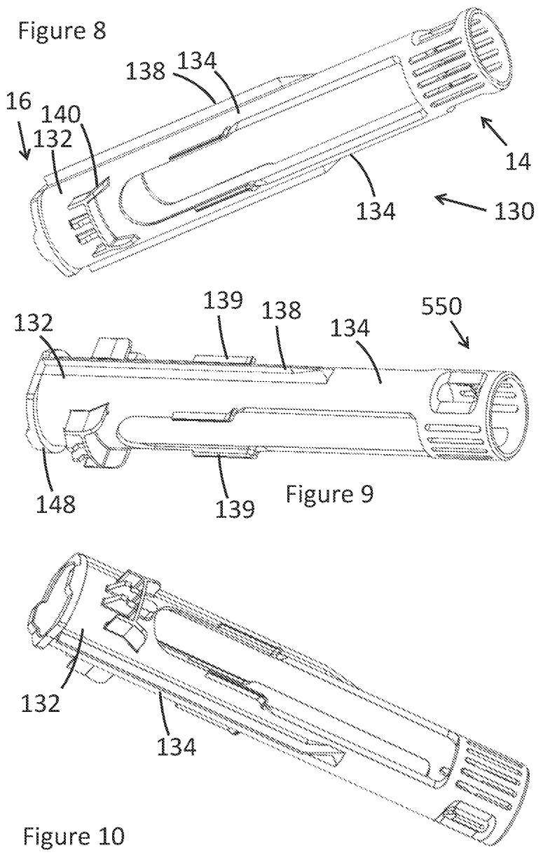

An example of a syringe carrier of this type is shown in FIGS. 8 to 12. As can be seen from FIGS. 8 to 10, the syringe carrier comprises an optional base 132 at the distal end of the syringe carrier, with two optional arms 134 extending in the proximal direction from the proximal end of the base 132. The proximal ends of the arms 134 are attached to a proximal portion 550 of the syringe carrier 130. The proximal portion 550 of the syringe carrier 130 is shown in more detail in FIG. 11. The proximal portion 550 of the syringe carrier comprises a first ring 552 (proximal ring), a second ring 554 (distal ring), and a plurality of strips 556 extending from the first ring 552 to the second ring 554. The first ring 552 is closer to the proximal end of the syringe carrier 130 than the second ring 554. The second ring 554 is attached to the arms 134. The strips 556 are flexible. Each flexible strip 556 comprises a protrusion 558 that extends from the strip towards the longitudinal axis. An optional cut-out 560 is also visible. The cut-out 560 can be beneficial for material reduction and/or as a support feature to help attach the syringe carrier to another medicament delivery device component such as a housing.

The protrusions extend from the strips towards the longitudinal axis (i.e. the protrusions are radially inwardly extending protrusions). The shape of the protrusions can be varied, for example to match the shape of a shoulder of a syringe. This can help spread the load of a syringe on the protrusion.

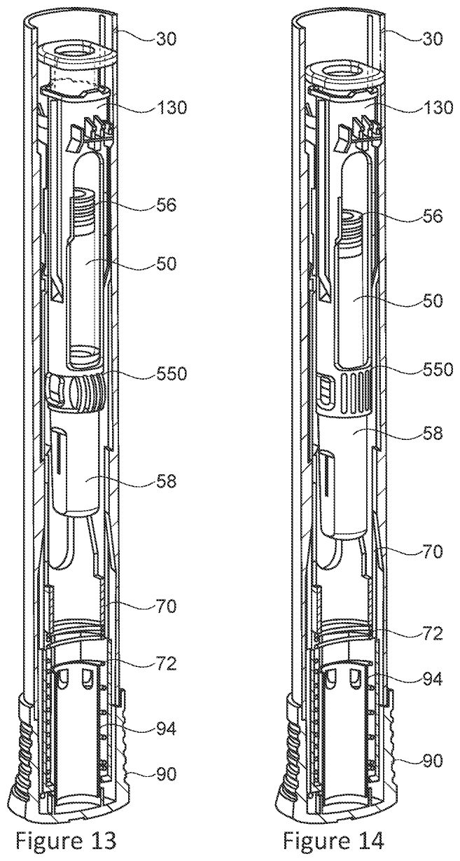

During assembly of a medicament delivery device (or a medicament delivery device sub-assembly) comprising a syringe carrier as described, a housing is first provided. The syringe carrier is then inserted into the housing. A syringe is then inserted into the syringe carrier (though the syringe carrier could alternatively be inserted into the housing after the syringe has been inserted into the syringe carrier). When the rigid needle shield encounters the proximal portion 550 of the syringe carrier, the rigid needle shield pushes the flexible strips 556 outwards away from the longitudinal axis to allow the rigid needle shield to pass (FIG. 13)(a close up of the proximal portion of the syringe carrier whilst in this position is also shown in FIG. 12). Specifically, the rigid needle shield pushes the protrusions outwards. Once the rigid needle shield has passed the protrusions, the protrusions can move back towards the longitudinal axis, thereby entering the gap between the rigid needle and a shoulder of the syringe (FIG. 14). In this position the protrusions can support the shoulder of the syringe.

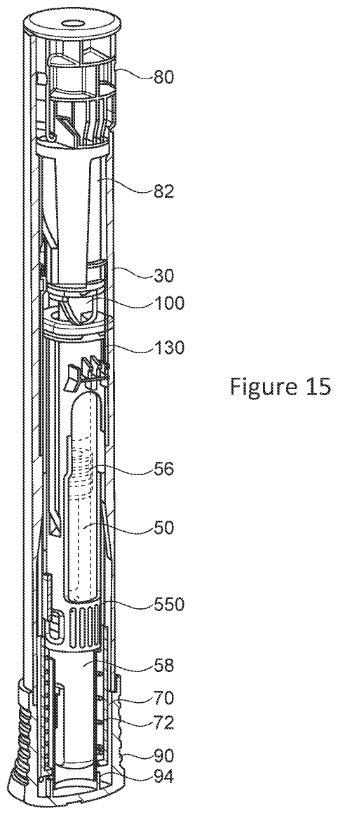

In this example these steps occur while the syringe carrier is in a distal position (interim position) relative to the housing (although these assembly steps could alternatively occur with the syringe carrier in a final position inside the housing). Subsequently, the syringe carrier is moved in the proximal direction from the distal position to a proximal position (final position). The final position of the syringe carrier is shown in FIG. 15. In FIG. 15, other features of an example completed medicament delivery device can also be seen.

The features shown in FIGS. 13 to 15 will now be briefly described. In addition to the housing 30, the syringe 50 and the syringe carrier 130, the autoinjector 10 comprises a powerpack (comprising a rear housing 80, a rotator 82 and a plunger rod 100, along with a plunger rod spring (not visible)), a cap 90 comprising a RNS remover 94 and an optional needle guard 70 and needle guard spring 72.

In this example, the base is tubular, which can be beneficial in terms of supporting a syringe, but is optional. Having a base is optional, as the functionality of supporting the syringe can be provided just by the proximal portion of the syringe carrier. The base could be attached directly to the proximal portion of the syringe carrier (i.e. without arms), but the provision of arms can be beneficial as it can allow for easy visibility of the medicament inside the syringe between the arms. Instead of two arms, one three, or more arms could be provided. The syringe carrier is shown with various supporting ribs, arms, protrusions and flanges which are described in more detail below. These optional supporting features are attached to the base and the arms in this example, although they could be additionally or alternatively attached to the proximal portion of the syringe carrier. These features can be beneficial in helping position the syringe carrier relative to other medicament delivery device components and/or to help attach the syringe carrier to other medicament delivery device components.

In this example, ten strips 556 are shown, grouped into two sets of five opposite one another. This arrangement can be beneficial as it can help evenly spread the load of supporting the syringe. Instead of this arrangement, more or fewer strips could be provided. For example, two or more strips could alternatively be provided, for example 4 or 8 strips. The strips could be evenly spaced out around the circumference of the proximal portion, instead of grouped into two sets. The strips could be split into three or more sets rather than two sets. Preferably, each individual strip has another strip directly opposite it relative to the longitudinal axis; this can help balance the forces on the syringe carrier. This is optional, however—with a syringe carrier with three strips (for example), the strips could be spaced apart by 120 degrees from each other around in the circumferential direction. The strips typically extend parallel to the longitudinal axis, though the strips could alternatively be angled relative to the longitudinal axis.

The syringe carrier would typically be a single integral part, though it could alternatively be two or more parts attached to one another, for example by glue or a snap fit.

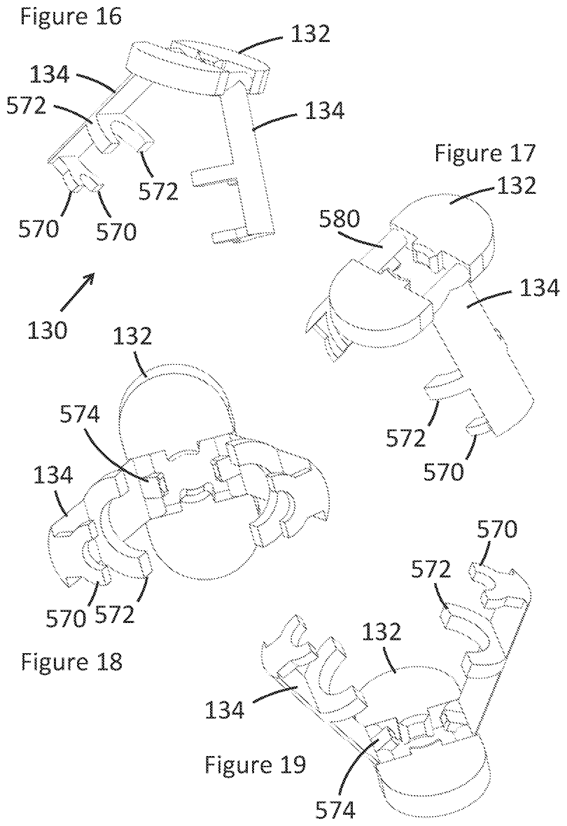

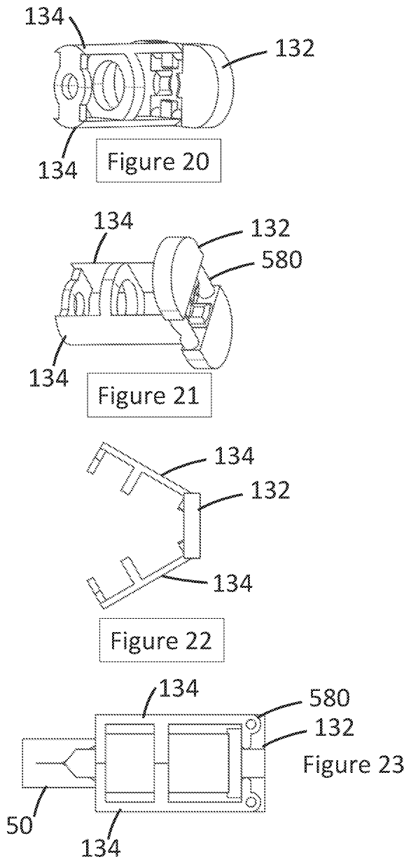

FIGS. 16 to 23 show another syringe carrier. FIGS. 16 to 19 and 22 show various views of the syringe carrier when open, FIGS. 20 and 21 show two views of the syringe carrier when closed, and FIG. 23 shows the syringe carrier with a syringe in the syringe carrier.

The syringe carrier comprises three parts, namely two arms 134 and a base 132. The two arms can pivot relative to the base, resulting in a closed position in which protrusions on the arms are touching one another (e.g. FIG. 20), and an open position in which the protrusions on the arms are spaced apart from one another (e.g. FIG. 16). In the open position, a syringe can be inserted into the syringe carrier (e.g. FIG. 22). In the closed position, a syringe can be held in the syringe carrier (e.g. FIG. 23).

In the depicted example, each of the two arms has two pairs of protrusions 570, 572, and when the syringe carrier is closed, the protrusions 570, 572 on the two arms combine to create two rings to hold the syringe carrier (as the protrusions extend in the circumferential direction relative to the axis—this can help evenly support the syringe. The proximal ring of the two rings (i.e. the two protrusions 570) supports the shoulder of the syringe, and the distal ring of the two rings (i.e. the two protrusions 572) supports the medicament holder of the syringe. The rotation axes of the hinges 580 where the arms pivot relative to the base are perpendicular to the longitudinal axis. Each of the arms also comprises an optional protrusion 574 adjacent to the pivot point and extending towards the longitudinal axis. These protrusions can be useful during assembly as outlined below, and can also help limit axial movement of the syringe after it is placed in the syringe carrier.

During assembly, the syringe carrier is first placed in the open position. The syringe is then inserted into the proximal end (though the syringe could alternatively initially be inserted from the side) and then moved in the distal direction. The syringe carrier is then closed. This closure can be achieved by pushing the syringe against the optional protrusions adjacent to the pivot point, which will pivot the arms towards the closed position (in the absence of these optional protrusions, the arms can be pushed into the closed position). The result is a sub-assembly with a syringe carrier and a syringe as shown in FIG. 23, which can subsequently be inserted into a medicament delivery device.

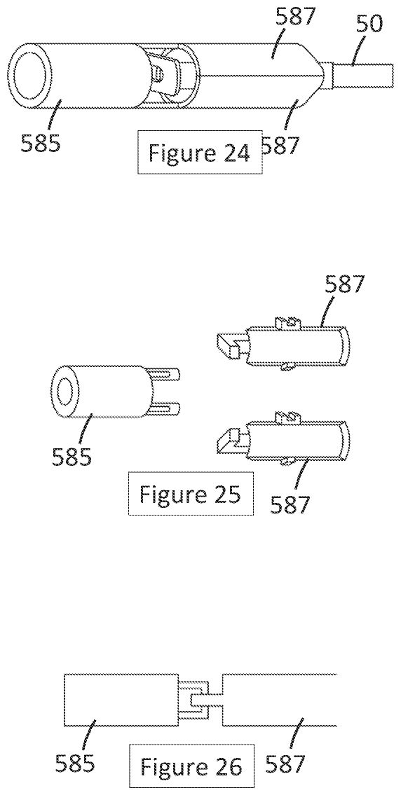

A second example is shown in FIGS. 24 to 26. In this example, the syringe carrier comprises three separate components, namely a base 585 (rear cap) and two body halves 587, as can be seen in FIG. 25 in particular. The proximal end of the two body halves 587 can support the syringe 50 at the shoulder (and optionally at the medicament holder as well by restricting radial movement of the syringe), and the base 585 can optionally support the distal end of the syringe (e.g. the flange). The body halves 587 can be attached to each other by snap fits or by gluing, for example.

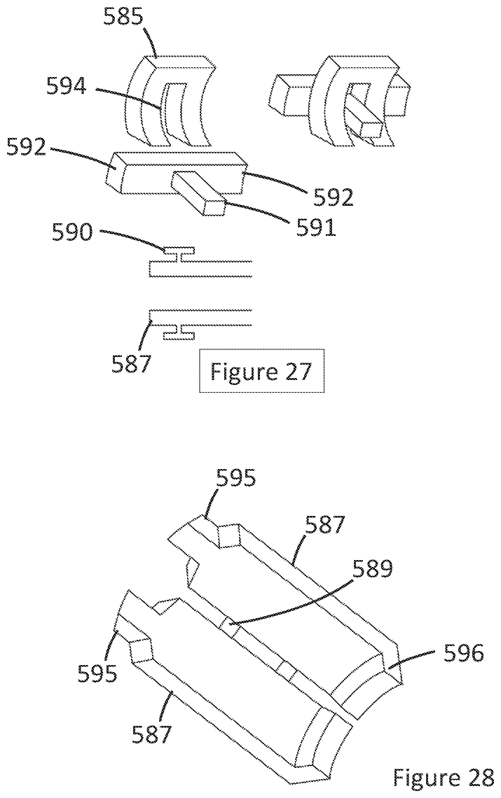

A third example is shown in FIGS. 27 and 28. This is a variation of the example in FIGS. 24 to 26, but this time the two body halves are connected together by a hinge 589 as shown in FIG. 28. The rotation axis of the hinge is parallel to the longitudinal axis of the syringe carrier. An alternative mechanism for attaching the base 585 to the body halves 587 is also shown in FIG. 27, where part of the base and part of the body halves are shown. A T-shaped protrusion 590 of a body half 587 engages a groove 594 on the base 585. Even though the base and the body halves are not necessarily fully fixed together by this arrangement, this arrangement restricts movement in the axial direction of the body halves relative to the base, which is enough to hold the base and body halves in place if radial movement relative to one another is restricted by other parts of a medicament delivery device (such as a housing). Only the trunk 591 of the T-shaped protrusion is necessary; the arms 592 are optional. Although not visible in FIG. 28, a T-shaped protrusion could be attached to each of the distal protrusions 595 of the body halve 587. A flange 596 can support a shoulder of a syringe; a similar flange could be provided for the example in FIGS. 24 to 26.

The examples herein focus on autoinjectors, but the examples described herein could be implemented in other medicament delivery devices more generally, such as in pen injectors. Some of the examples herein focus on 1 ml syringes, but the designs described herein could also be used on other volumes and other types of medicament container, for example a syringe without an attached needle rather than a syringe with an attached needle. An example of an autoinjector 10 that could comprise the syringe carriers described herein is shown in FIG. 29. The example autoinjector extends along an axis 12 in an axial direction 13 between a distal end 15 and a proximal end 14, with a radial direction 17 and a circumferential direction 16 also depicted for reference. A housing (or body) 30 and a cap 90 of the autoinjector can be seen, along with an optional window 32 in the body. The autoinjector can house a syringe. The autoinjector typically includes features such as a powerpack and a needle guard inside the housing. The shape of the housing and of the cap could be varied from those shown in the example—for example, the housing could be triangular in cross section perpendicular to the axis rather than circular, could be an irregularly-shaped tube rather than a cylinder, and/or the housing could be two or more components rather than a single component. The autoinjector shown does not have an activation button, though one could be provided (i.e. a three-step autoinjector rather than a two-step autoinjector).

FIGS. 30 and 31 show an example of a syringe for reference. This particular syringe 50 comprises a medicament holder (medicament container) 52, a needle 54, a stopper 56, a rigid needle shield (RNS) 58, a flexible needle shield (FNS) 60, a flange 62 and a shoulder 64. The syringe extends from a proximal end 14 to a distal end 15. The medicament holder 52 is tubular (specifically cylindrical in this example), with the flange 62 at the distal end of the medicament holder 52 and the needle 54 at the proximal end of the medicament holder 52. The stopper 56 is in the medicament holder 52. The flexible needle shield 60 extends around the needle 54, and the rigid needle shield 58 extends around the flexible needle shield. The shoulder 64 is the proximal end of the medicament holder 52. One particular example syringe is described here, but other syringes could be used. For example, a needle 54 is included in examples described herein, but other medicament delivery members such as jet injectors could alternatively be used, or the needle could be provided separately rather than as an integral part of the syringe. A needle shield comprising a rigid needle shield 58 and a flexible needle shield 60 is included in examples described herein, but the examples described herein could be used with needle shields without a flexible needle shield or even entirely without a needle shield, although the examples described herein can be particularly beneficial when used with syringes with an RNS. The flange 62 is optional. The syringe could be various sizes, including but not limited to 1 ml and 2.25 ml.

Example mechanical powerpacks are described herein (for example the powerpack shown in FIG. 15), but other types of powerpack could be used instead, for example an electrically powered powerpack or a gas-powered powerpack. An example of a device in which syringe carriers as described herein could be used is provided in WO2011/123024, which is hereby incorporated by reference.

A base 132 is described herein. Typically, the base is depicted as the distal portion of the syringe carrier, but could alternatively be spaced apart in the axial direction from the distal end of the syringe carrier.

Arms 134 are described herein. Most (though not all) of the examples herein use two arms, although in the examples with two arms, one, three or more arms could alternatively be provided. Similarly, other features that are provided (other arms, protrusions, cut-outs, apertures, recesses and the like) can generally be provided in a quantity different to the specific number described in the examples given.

Many of the syringe carriers described herein comprise some kind of optional protrusion or rib. For example, the syringe carrier shown in FIGS. 8 to 10 comprises two ribs 138, four second ribs 139 and two protrusions 140. The ribs 138 extend in the longitudinal direction, with the proximal end of each rib 138 attached to an arm 134 and the distal end of each rib 138 attached to the base 132. The second ribs 139 extend in the longitudinal direction. A second rib 139 is arranged on each side of each arm 134 in the circumferential direction. The protrusions 140 are attached to the base 132. Another optional feature is a distal flange 148, which is also present in many of the syringe carriers described herein. The flange 148 is attached to the base 132 and extends in the radial direction away from the axis and extends in the circumferential direction around the base. These ribs, protrusions and flanges can provide various advantages, including helping to align the syringe carrier relative to other features during assembly, maintaining rigidity of the syringe carrier, and/or fixing the position of the syringe carrier relative to other components of a medicament delivery device in a completed device. Whilst these protrusions 140, ribs 138, 139 and flanges 148 are depicted as having a particular shape, these shapes could be varied depending on factors such as the desired rigidity and on the shape of other components within a particular design of medicament delivery device.

In the present disclosure, when the term “distal direction” is used, this refers to the direction pointing away from the dose delivery site during use of the medicament delivery device. When the term “distal part/end” is used, this refers to the part/end of the delivery device, or the parts/ends of the members thereof, which under use of the medicament delivery device is/are located furthest away from the dose delivery site. Correspondingly, when the term “proximal direction” is used, this refers to the direction pointing towards the dose delivery site during use of the medicament delivery device. When the term “proximal part/end” is used, this refers to the part/end of the delivery device, or the parts/ends of the members thereof, which under use of the medicament delivery device is/are located closest to the dose delivery site.

Further, the terms “longitudinal”, “longitudinally”, “axially” and “axial” refer to a direction extending from the proximal end to the distal end and along the device or components thereof, typically in the direction of the longest extension of the device and/or component. The circumferential direction describes a direction extending around the axis, so around the circumference of a device or component, and the radial direction extends perpendicular to the axis.

Similarly, the terms “transverse”, “transversal” and “transversally” refer to a direction generally perpendicular to the longitudinal direction.

Generally, all terms used in the claims are to be interpreted according to their ordinary meaning in the technical field, unless explicitly defined otherwise herein. All references to a/an/the element, apparatus, member, component, means, etc. are to be interpreted openly as referring to at least one instance of the element, apparatus, member component, means, etc., unless explicitly stated otherwise.

Various modifications to the embodiments described are possible and will occur to those skilled in the art without departing from the invention which is defined by the following claims.

Some aspects of the invention are summarised in the following clauses.
1. A syringe carrier (130) for a medicament delivery device (10), wherein the syringe carrier (130) is tubular and extends along a longitudinal axis from a proximal end to a distal end, the syringe carrier (130) comprising a first ring extending around the longitudinal axis, a second ring extending around the longitudinal axis, and a plurality of flexible strips extending from the first ring to the second ring, wherein the first ring is closer to the proximal end of the syringe carrier than the second ring, and wherein each flexible strip comprises a protrusion that extends from the strip towards the longitudinal axis.
2. The syringe carrier of clause 1, wherein the plurality of flexible strips extend parallel to the longitudinal axis.
3. The syringe carrier of clause 1 or 2, wherein the plurality of flexible strips each extend from a first end to a second end, and wherein each of the plurality of flexible strips is attached to the first ring at the proximal end of the flexible strip and is attached to the second ring at the distal end of the flexible strip.
4. The syringe carrier of any previous clause, wherein the plurality of flexible strips are parallel to one another.
5. The syringe carrier of any previous clause, wherein the plurality of flexible strips is one or more pairs of flexible strips, and wherein the two strips of each pair of flexible strips are opposite one another relative to the longitudinal axis.
6. The syringe carrier of any previous clause, wherein the first ring is at the proximal end of the syringe carrier.
7. The syringe carrier of any previous clause, wherein the syringe carrier comprises a base at the distal end of the syringe carrier and two arms extending in the proximal direction from the base, and wherein the second ring is attached to the proximal end of each of the two arms.
8. The syringe carrier of any previous clause, wherein the syringe carrier is a single integral part.
9. A medicament delivery device sub-assembly comprising a syringe carrier and a syringe inside the syringe carrier, the syringe comprising a rigid needle shield and a medicament container, wherein the protrusion of the flexible bridge is in a gap between the rigid needle shield and a shoulder of the medicament container.
10. A medicament delivery device comprising the syringe carrier of any of clauses 1 to 8 or the sub-assembly of clause 9.
11. A method of assembling a sub-assembly of a medicament delivery device, the method comprising the steps of:
   providing a housing (30), a syringe carrier (130) and a syringe (50) according to any of clauses 1 to 8, wherein the housing (30) extends in a longitudinal direction along a longitudinal axis (12) from a proximal end to a distal end;
   inserting the syringe carrier (130) into the housing (30) so that the syringe carrier (130) is aligned with the housing (30) along the longitudinal axis (12); and inserting the syringe (50) into the syringe carrier (130) in the longitudinal direction so that the plurality of flexible strips of the syringe carrier move away from the longitudinal axis to allow a rigid needle shield of the syringe to pass.
12. A syringe carrier for a medicament delivery device, wherein the syringe carrier comprises a base and two arms, the arms each being pivotally attached to the base, the arms being configured to pivot from a closed position in which the arms are in contact with one another to an open position in which the arms are spaced apart from one another.
13. A syringe carrier for a medicament delivery device, wherein the syringe carrier comprises two body halves, wherein the body halves are configured to enclose a syringe within the two body halves when the two body halves are attached to one another.
14. A syringe carrier, wherein the syringe carrier comprises a flexible portion arranged between a proximal end of the syringe carrier and a distal end of the syringe carrier, and wherein the flexible portion comprises a bridge and a protrusion.

1-23. (canceled)

24. A medicament delivery device sub-assembly, the medicament delivery device sub-assembly comprising:
a housing extending along a longitudinal axis from a proximal end to a distal end, the housing comprising a tubular section with an internal surface facing towards the axis and an external surface facing away from the axis, wherein the housing comprises an aperture extending through the tubular section from the internal surface to the external surface; and
a housing cover assembly attached in the aperture of the housing, the housing cover assembly being configured to hold a syringe in place relative to the housing.

25. The medicament delivery device sub-assembly of claim 24, wherein the housing cover assembly comprises a protrusion extending towards the longitudinal axis.

26. The medicament delivery device sub-assembly of claim 25, wherein the protrusion of the housing cover assembly comprises a surface facing the distal end of the housing, wherein the surface is shaped to fit between a medicament delivery shield and a shoulder of said syringe so as to support said shoulder of said syringe.

27. The medicament delivery device sub-assembly of claim 24, wherein the medicament delivery device sub-assembly comprises a syringe in the housing, and wherein the syringe is held relative to the housing by the housing cover assembly.

28. The medicament delivery device sub-assembly of claim 24, wherein the housing cover assembly consists of two housing covers arranged opposite each other relative to the axis.

29. The medicament delivery device sub-assembly of claim 24, wherein the housing cover assembly comprises two separate housing covers.

30. The medicament delivery device sub-assembly of claim 24, wherein the housing cover assembly is a single integral part.

31. The medicament delivery device sub-assembly of claim 24, wherein the housing cover assembly comprises an arm.

32. The medicament delivery device sub-assembly of claim 24, wherein the arm extends in the direction of the axis.

33. The medicament delivery device sub-assembly of claim 24, wherein the arm is rigid.

34. The medicament delivery device sub-assembly of claim 25, wherein the housing cover assembly comprises an arm, where the protrusion of the housing cover assembly is attached to the arm.

35. The medicament delivery device sub-assembly of claim 24, wherein the external surface of the tubular section is configured to be held by a user during use of a medicament delivery device comprising the medicament delivery device sub-assembly.

36. The medicament delivery device sub-assembly of claim 24, wherein the housing cover assembly comprises a window.

37. The medicament delivery device sub-assembly of claim 36, wherein the housing cover assembly comprises a window frame extending around the window.

38. The medicament delivery device sub-assembly of claim 24, wherein the housing cover assembly comprises at least one pin extending perpendicular to the longitudinal axis.

39. The medicament delivery device sub-assembly of claim 38, wherein the pin is arranged so that the pin is between a syringe and a syringe carrier in a completed medicament delivery device.

40. The medicament delivery device sub-assembly of claim 39, wherein the pin is arranged so that the pin is between a shoulder of the syringe and the syringe carrier in a completed medicament delivery device.

41. The medicament delivery device sub-assembly of claim 24, wherein the housing cover assembly comprises an insert and the housing comprises a flexible arm adjacent to the insert.

42. The medicament delivery device sub-assembly of claim 24, wherein the housing cover assembly is configured to indirectly hold a syringe in place relative to the housing.

43. A medicament delivery device comprising:
a housing extending along a longitudinal axis from a proximal end to a distal end, where the housing comprises a tubular section with an internal surface facing towards the axis and an external surface facing away from the axis;
an aperture extending through the tubular section from the internal surface to the external surface;
a syringe carrier axially fixed within the housing;
a syringe positioned within the syringe carrier and having a shoulder located at a proximal end of the syringe; and
a housing cover assembly attached in the aperture of the housing, where the housing cover assembly engages the shoulder to axially fix the syringe relative to the housing.
